(19) United States
(12) Patent Application Publication
(10) Pub. No.: US 2013/0164776 A1
(43) Pub. Date: Jun. 27, 2013

(54) METHOD USED IN A HUMAN OR ANIMAL FAECES SAMPLE PROCESSING SYSTEM, AND A SAMPLE PROCESSING SYSTEM

(71) Applicants: Lars Olof A Hansson, Stocksund (SE); Enar Bernt Bernt Johansson, Lammhult (SE)

(72) Inventors: Lars Olof A Hansson, Stocksund (SE); Per Spangeus, Lammhult (SE)

(21) Appl. No.: 13/511,340

(22) PCT Filed: Nov. 17, 2010
(86) PCT No.: PCT/SE2010/051267

§ 371 Date: Sep. 6, 2012

(30) Foreign Application Priority Data
Nov. 23, 2009 (SE) 09508888

(51) Int. Cl. C12Q 1/04

(52) U.S. Cl. CPC C12Q 1/04
USPC 435/34; 422/547; 422/533

(57) ABSTRACT

A sample processing system, and a method in relation thereto, adapted to automatically process a sample, e.g. a faecal sample, prior to the analysis of the sample. The system comprises a sample container for collecting a sample, and a control unit comprising a stored processing recipe that includes parameters to be used to automatically perform the processing of the sample. The control unit is adapted to generate robot control signals, obtained from said processing recipe, to be applied to a robot arranged to move the sample container between different units of the system and to generate control signals to a specific unit when the sample container is about to be treated by that unit. The robot is arranged to automatically move the sample container to: a weighing unit (optional) to determine the weight of the sample; a buffer supply unit to add buffer where the buffer amount can be predefmed or depend on the weight of the sample; a homogenization and mixing unit to homogenize and mix the sample in the buffer, preferably by using sonication by ultrasound. In addition the sample container is preferably moved to: a centrifuge to centrifuge the container in order to produce a clear supernatant; a transfer unit in order to transfer a part of the supernatant to a secondary container, the transferred volume is predefined, or related to the weight of the sample and/or related to the amount of buffer previously added; and if needed, the buffer supply unit adds buffer to the secondary container in order to dilute the sample solution to the desired final concentration.

### FIELD OF THE INVENTION

The present invention relates to a method and a system according to the preambles of the independent claims.

### BACKGROUND OF THE INVENTION

Since a long time, analysis (assay) development in clinical laboratories has focused on finding new markers in blood and urine, but not in faeces (stool). Faecal analysis (assay) has shown slow development due to the difficult nature of the sample, and inconvenience for laboratory staff of sample handling. Faecal testing has been restricted to look for virus, bacteria and traces of blood. In recent years faecal sample analysis has received high attention, resulting in the development of important diagnostic markers in faeces such as calprotectin for inflammatory bowel disease as well as various cancer markers. This drives a fast growth in the number of faecal samples to be analysed, which in turn requires more efficient systems for sample handling.

Calprotectin is a strong faecal marker for inflammatory bowel disease (IBD =Crohn's disease and ulcerative colitis) affecting ˜1% of the population. Diagnosis of IBD is today mainly done by colonoscopy. Also, calprotectin is used to differentiate IBD and the benign IBS (colon irritable) that affects ˜15% of the population. Furthermore, faecal calrotectin is also used to monitor the effect of treatment and for detection of relapses of IBD. Use of calprotectin can reduce colonoscopy by 75% in IBD and 100% in IBS. Calprotectin is a cheap and patient friendly test compared to the costly and for the patient inconvenient method of colonoscopy. This drives a strong growth in calprotectin analyses done by clinical laboratories. Also, there is a high volume of faecal tests done in microbiology laboratories as well, detecting molecular biological markers for virus and bacteria. In the future, faecal analysis will also be used to find genetic and biological markers for gastro-intestinal cancers.

Today, a manual process is needed for faecal samples prior to analysis, which is illustrated in FIG. 1. The present manual pre-processing of faecal samples, prior to analysis of calprotectin, comprises several steps, such as extraction of the right amount of sample from the faecal container, weighing the sample, e.g. 50-150 mg faecal sample (FIG. 1a), addition of appropriate buffer (FIG. 1b), mixing of solution (by vortex and shaking), e.g. with a vortex for 60 seconds and then shake vigorously for 20 minutes (FIG. 1c), decant to a centrifuge-tube, centrifugation for 5 minutes (FIG. 1d), and transfer of supernatant after centrifugation to a secondary container (FIG. 1e). This manual faecal sample processing is time consuming, has low throughput and is very inconvenient for laboratory staff both because of the smell but also for the risk of spread of infectious agents and risk for occupational (work-place) injury. Furthermore, manual processing yields high variation in the quality of the processed sample, which implies unnecessary high variations in the subsequent analytical results. Our invention solves all of these problems.

Presently, there are no automatic systems on the market to solve the pre-processing problem. The only alternative is manual pre-processing of faecal samples (FIG. 1). The switch to automation is competitive cost-wise, since the cost of automation is balanced by cost savings through substantially less need of personnel resources (less hands-on time). Thus, strong drivers for the method and system according to the present invention are the cost savings, the improved working environment for staff, and a higher analytical quality. Also, an automatized process yields a higher throughput capacity than the manual process.

We also predict that there are potential for the invention in markets other than pre-processing of human faecal samples. Such areas are:
Veterinary Medicine, to pre-process animal faeces
Microbiology, to pre-process samples before analysis of bacterial, viral or parasitic biomolecules
Tissue sample pre-processing
Tobacco industry, to pre-process tobacco products for content analysis
Pharma industry, to pre-process pharmaceuticals such as pills for analyzing content
Food industry, to pre-process food items for analyzing content

In clinical settings, microbiology samples are taken, preferably using a swab or a brush. The goal is to detect the presence of bacteria, virus, fungi or parasities either through the intact organism or through different biomolecules such as DNA, RNA or an antigen. The sample is normally taken from a wound, or the surface or a cavity of a body or from a stool sample. At the laboratory, when the aim is to detect biomolecules, the microbes in the sample are lysed using chemicals, mechanical tearing or ultrasound sonication to enable downstream analysis of the respective biomolecules. Ultrasound has proved to be a strong tool for lysing microbes. Today antibiotic resistant bacteria such as MRSA are a big problem in healthcare and there is a need for fast and high-throughput detection of MRSA. MRSA can be detected through analysis of DNA/RNA. For the actual DNA/RNA analysis; comprising DNA/RNA extraction, amplification and detection; there are fast and high-throughput systems, the problem is the slow and cumbersome lysing and centrifugation pre-processing steps before the analysis. We address this problem with our invention. This pre-processing invention can be used throughout clinical microbiology when the intention is to detect bacterial, viral or fungal biomolecules. Another application in microbiology that needs fast detection is to detect a genital Group B Streptococcus (GBS) infection in a pregnant woman, prior to giving birth.

Below, some prior art documents are shortly listed that relate to different aspects of sample processing. However, these documents are only remotely related to the invention.
EP-2,088,418 relates to a method for the integrated and automated analysis of biological samples, e.g. faecal samples. Related to the initial steps of the procedure, i.e. sampling and handling of a sample prior to analysis.
U.S. Pat. No. 7,521,023 relates to an apparatus and methods for controlling sonic treatment and/or controlling the location of a sample relative to the sonic energy.
WO-98/20355 relates to a manual process for the extraction of proteins from gastrointestinal tract samples taken from humans or other mammals.
U.S. Pat. No. 4,835,707 relates to an automatic analysis method and apparatus for enzyme reaction.

Thus, the objective of the present invention is to achieve a more efficient system to automate the pre-process of samples, and in particular faecal, tissue or microbiological samples.

### SUMMARY OF THE INVENTION

The above-mentioned objective is achieved by the present invention according to the independent claims.

Preferred embodiments are set forth in the dependent claims.

The present invention is an automatic method and system for automatic pre-analytical processing, in particular of faecal samples. This pre-processing is today done manually. The automatic process according to the present invention comprises identification and registration of the sample and the sample-tube, weighing the sample, adding appropriate buffer volume, ultrasound sonication to homogenize and mix the sample in the buffer, followed by centrifugation and transferring the supernatant for subsequent analysis. The whole automatic process is unique.

In particular the present invention is adapted for clinical use of automatic pre-processing faecal samples for subsequent calprotectin analysis.

The current manual faecal pre-processing is time consuming, low throughput and unpleasant for laboratory staff and induces risks for both of spread of infectious agents and for occupational (work-place) injury. The automatic pre-process improves the throughput and working environment for the staff. Furthermore, it has been observed that the method and system according to the present invention enhances the quality of the extraction procedure and thus yields more consistent calprotectin assay results.

The automatic processing system may be used in medical laboratories all over the world; both clinical chemistry units (e.g. doing calprotectin analysis) and microbiological units (e.g. doing analysis for viruses, bacteria, etc.). It can also be used in other areas, as described in the background part of this document.

A (pre-weighted) sample container is used to collect the sample prior to pre-processing. In the case of faecal samples, the sample container is preferably made of plastics. As the collection of faeces is normally done by the patient, it must be convenient and hygienic to use, as well as yield the appropriate amount of sample.

### SHORT DESCRIPTION OF THE APPENDED DRAWINGS

FIG. 1 is a schematic block diagram illustrating the today manual pre-analytical sample handling.

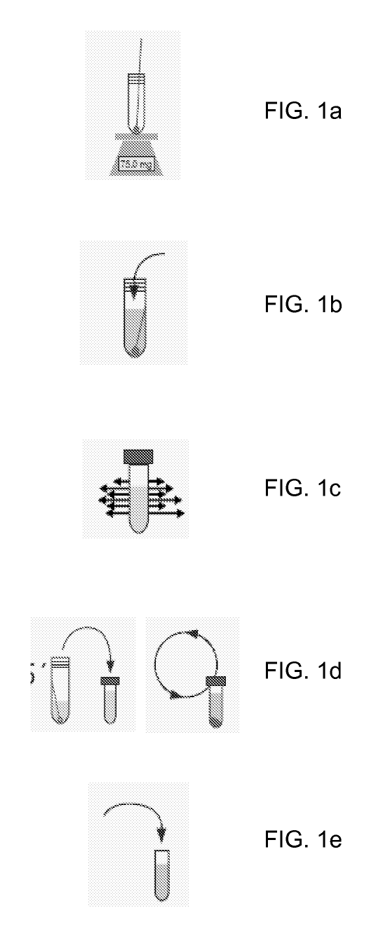

FIG. 2 is a schematic block diagram illustrating the system according to the present invention.

FIG. 3 is a schematic illustration of a sample container that may be used in connection with the method and system of the present invention.

FIG. 4 is a flow diagram of the method according to the present invention.

### DETAILED DESCRIPTION OF PREFERRED EMBODIMENTS OF THE INVENTION

With references to the figures the invention will now be described in detail.

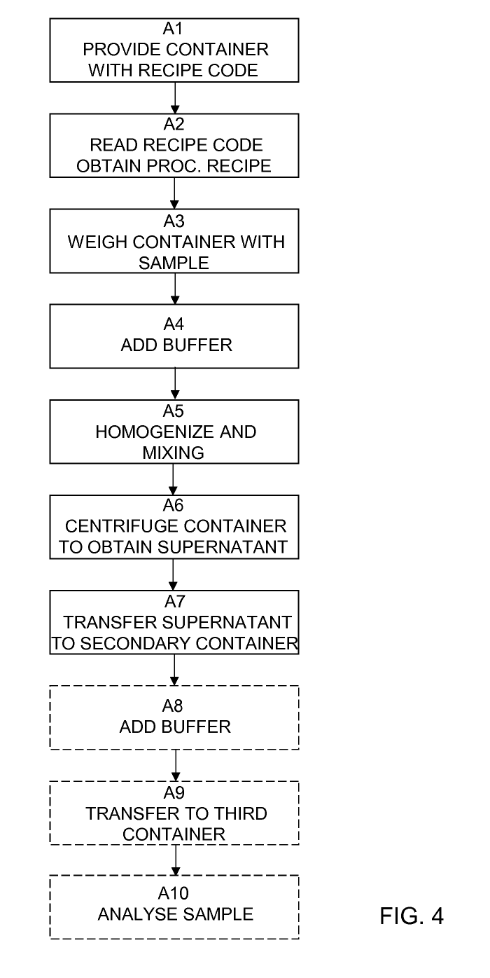

FIG. 4 illustrates a method according to the present invention of automatic processing of a sample collected in a sample container, to be performed prior to the analysis of the sample. In FIG. 4 optional steps are illustrated by dashed boxes.

A specific sample container is used to collect the sample, e.g. human or animal faeces, or human or animal tissue, tobacco, food products, or pharmaceuticals. This container may be pre-filled with a buffer to enhance the sample stability and/or to enhance the subsequent extraction of the analyte. The biological sample, e.g. a faecal sample, is collected and put into the sample container. In the case of faecal samples usually a plastic device is used for sampling. Then the sample (with or without the device included) is put into the sample container, which is then closed and sent to the laboratory. Sample containers are commercially available today but can also be specially designed if needed. A buffer may also be manually added to the sample container after the sample has been collected in the container.

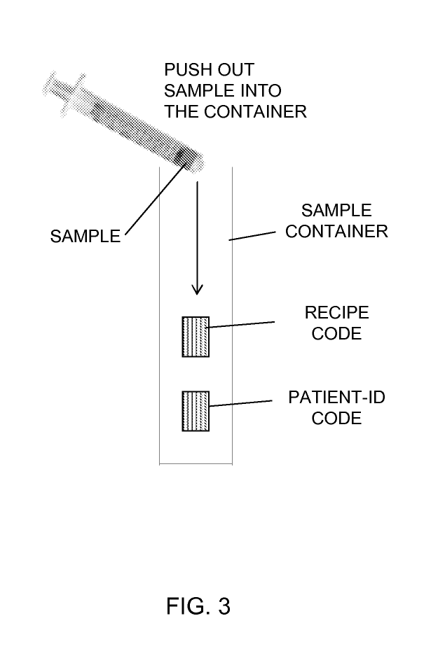

FIG. 3 illustrates an example of a sample container including a sampling device. The sampling device is a kind of syringe used to collect the sample. The sample is then displaced into the sample container by pushing it out from the syringe.

The method (see FIG. 4) comprises a process recipe related to the sample. The recipe includes parameters required for the sample processing steps to be performed by a robot. The method comprises of the following automatically performed processing steps:

A1: providing the sample container with a recipe code and a patient-ID code being arranged in connection to the sample container.

A2: reading the recipe code and a patient-ID code of the sample container to obtain the relevant information for processing the sample and registration of the patient.

The whole or parts of the processing recipe may be included in the recipe code, or as an alternative, the recipe code includes a pointer used to identify the relevant processing recipe stored in a control unit.

According to one embodiment the recipe code on the sample container is encrypted, and is decrypted in A2.

The sample container with a biological sample is put on the processing system which automatically reads the recipe code on the container. The recipe code e.g. identifies the specific process to be used for this specific sample.

In A2 the recipe code preferably is optically or electronically read. The recipe code may be a bar code (optically read) or RFID tag (electronically read). The sample container is probably sealed when it is put into the processing system, and must therefore be opened in order to gain access to the sample. This is automatically performed by the processing system before processing steps requiring direct access to the sample are performed, i.e. at least before step A4.

A3: weighing the sample container to determine the weight of the sample. The sample container is automatically weighed by a weighing unit to determine the amount of sample in the container. For faecal samples a normal range is 25-200 mg of faeces. Two process examples will be detailed herein; example 1 with a collected sample of 50 mg faeces and example 2 with a collected sample of 100 mg faeces. In the examples it is assumed that a final concentration of 0.4 mg sample/mL is desired.

A4: adding a buffer.

A buffer supply unit automatically adds a buffer. The buffer volume is predefined or related to the weight of the sample in step A3.

For instance, the buffer can be a tris-buffer, pH 7.4, with 0.154 mol NaCl which is a standard buffer commonly used today. The buffer can contain proteolytic inhibitors, e.g. benzamidine and/or EDTA, to improve sample stability.

For faecal samples, the buffer volume normally varies between 2 and 8 mL.

Example 1: Add 5 ml buffer to the 50 mg faeces.

Example 2: Add 5 ml buffer to the 100 mg faeces.

A5: homogenize and mix the sample in the buffer, preferably by using ultrasound sonication.

A homogenizing and mixing unit automatically homogenizes and mixes the biological sample in the buffer, preferably by using ultrasound sonication.

In the manual handling process for faecal samples today, one normally uses both a vibrating mixing device (vortex mixer) and a shaking mixer device for homogenization and mixing. However, the inventors have found that sonication gives a significantly better extraction recovery, is faster, and is easier to integrate in an automated solution. Of course, a vibrating and/or shaking device may be integrated into an automated solution, in accordance with the present invention, but this will increase the processing time and be significantly less efficient in extraction recovery.

According to one embodiment the steps A1-A5 are followed by the following steps A6-A8 being automatically performed. In an alternative embodiment, at least one of the steps A6-A8 is instead manually performed or performed on another automatic system.

A6: transferring the sample container to a centrifuge, and centrifuge the container in order to produce a clear supernatant.

The container with the homogenized and mixed sample and buffer is automatically transferred to a centrifuge in the system and then normally centrifuged for less than 5 minutes. The purpose of centrifugation is to produce a clear supernatant that will be used for subsequent analysis.

A7: transferring, by a transfer unit, a part of the supernatant to a secondary container. The transferred supernatant volume is predefined or related to the weight of the sample in A3, and may also be related to the volume of the added buffer in A4.

Example 1: Transfer 20 μL of the supernatant to the secondary container.

Example 2: Transfer 20 μL of the supernatant to the secondary container.

A8: if needed, add buffer to the secondary container in order to dilute the sample to the desired final concentration.

If needed, add buffer to the secondary container in order to dilute the sample to the desired final concentration. The amount of buffer can be predefined or related to the weight of the sample in A3, and may also be related to the volume of the added buffer in A4, and the amount of supernatant obtained from A7.

Since the weight of the sample can vary in A3, as well as the volume of the added buffer in A4, and the volume transferred in A7, this step ensures that the final concentration of the processed sample is within a certain range. Normally the concentration has to be within a certain range to match the requirement of a subsequent analytical system.

Example 1: Add 0.48 mL buffer to reach the desired concentration of 0.4 mg sample/mL.

Example 2: Add 0.98 mL buffer to reach the desired concentration of 0.4 mg sample/mL.

Although steps A1-A8 are presented in a logical succession, the order between some of the steps may be altered within the scope of the invention as defined by the appended claims. E.g. the buffer in step A4 may be added before the sample is weighed in step A3. Within the scope of the invention and its embodiments, one or more automatic steps may be added in between A1-A8 (whichever of these are applicable), such as transferring the whole or part of the sample solution to a new container to be processed by the next step in the method.

In a further embodiment the following step may be automatically performed after A8:

A9: transferring a part of the solution in the secondary container to a third container for further dilution by a buffer.

Thus, if needed, a part of the solution in the secondary container is automatically transferred to a third container by a transfer unit for further dilution by a buffer (as in A8). The amount of buffer can be predefined or related to the weight of the sample in A3, as well as to the amount of dilution already performed in previous buffer adding steps and to the amount of supernatant transferred in A7. The reason for step A9 may be that the desired concentration range was not reached in A8, and then this additional dilution is needed. Naturally, further dilutions may also be made if considered required.

According to a preferred embodiment the method comprises the following step to be performed after A8 or A9, whichever is applicable:
A10: analysing the sample.

The sample in the final container (the secondary or third container) is manually or automatically analyzed for one or more biomarkers. Automatic analysis can take place on the same robotic platform as the processing described above, or on a second analytic system to which the final container is manually or automatically transferred.

The analysis is normally performed on a separate analytical system, but it can also be integrated into the system that performs A1-A8 (A9). Typical types of analyses in faecal samples are immunological quantitative determination of peptides and proteins such as calprotectin, HNL, myeloperoxidase, alpha-1-antitrypsin, albumin, hemoglobin, and different tumour markers. Another group of analytes in faecal samples are DNA and RNA of either human or microbiological origin, e.g. to detect tumours or infection by virus or bacteria.

A non-exhaustive summary of information that may be included in the recipe code is listed below:
A statement of the sample type, e.g. a faecal sample
The weight of the sample container before the sample was added, e.g. used to calculate the weight of the sample in step A3
The size of the sample container, e.g. to optimally position the ultrasound probe in A5
The amount or/and type of buffer to add in A4
The time for the homogenization and mixing in A5 of the sample in the buffer
The centrifugation time in A6
The amount of supernatant to withdraw in A7
The amount or/and type of buffer to add in A8
The parameters for the dilution(s) in A9
Type of analysis to be done on the sample after processing, e.g. calprotectin, DNA or RNA

For instance, the recipe code for processing a faecal sample can be different from the recipe code for processing a tissue sample; or the recipe code to process a faecal sample can be different for analysing a protein biomarker vs. a genetic marker.

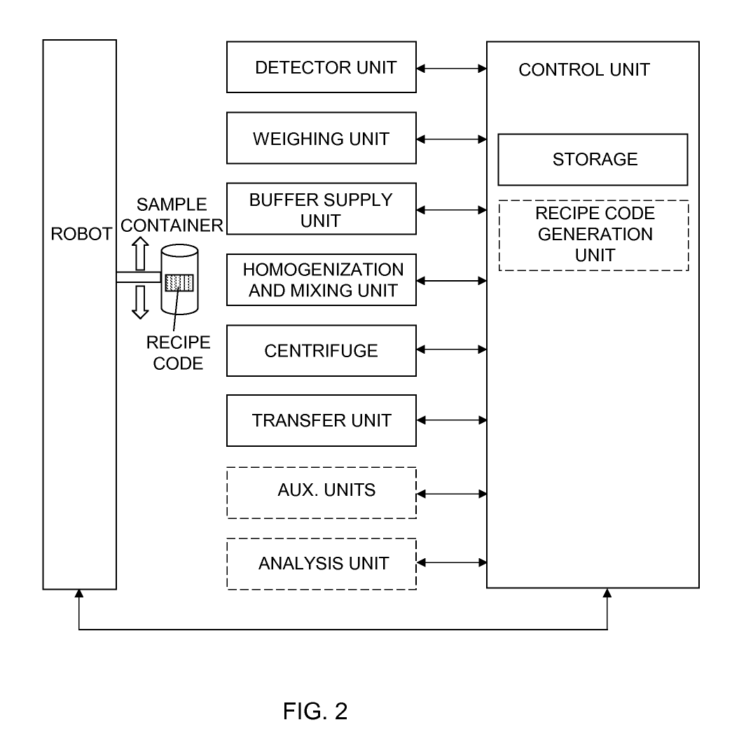

FIG. 2 describes a schematic illustration of a human or animal faeces sample processing system according to the present invention. The sample processing system is adapted to process a human or animal faeces sample prior to the analysis of the sample; comprising a sample container for collecting a sample; a control unit, e.g. a personal computer, or any dedicated device, comprising a storage including a processing recipe, stored therein, where the recipe contains parameters to be used to automatically perform the processing of the sample. The control unit is adapted to generate control signals to the robot, obtained from the processing recipe, to be applied to a robot arranged to move the sample container between different units of the system and also to generate control signals to a specific unit when the sample container is about to be treated by that unit.

The robot is arranged to automatically perform the following steps:
move the sample container to a weighing unit to determine the weight of the sample;
a buffer supply unit will add buffer to the sample, where the buffer amount can be predefined or depend on the weight of the sample;
move the sample container to a homogenization and mixing unit to homogenize and mix the sample in the buffer, preferably by using sonication by ultrasound.

If the sample container is sealed it must be opened, and this must be done at least before the buffer is added to the sample container. The opening of the sample container is naturally also an automatic step performed by the robot in dependence of appropriate robot control signals.

According to a preferred embodiment the system further comprises:
a centrifuge to centrifuge the container in order to produce a clear supernatant;
a transfer unit in order to transfer a part of the supernatant to a secondary container, the transferred volume is predefined, or related to the weight of the sample and/or related to the amount of buffer previously added; and if needed, the buffer supply unit adds buffer to the secondary container in order to dilute the sample solution to the desired final concentration, the buffer amount can be predefined, or depend on the weight of the sample and/or related to the amount of buffer previously added and/or related to the amount of supernatant previously transferred.

In one embodiment the buffer unit and the transfer unit are a single unit, often denoted the pipetting unit.

A further embodiment of the system comprises a recipe code generation unit adapted to provide said sample container with a recipe code, encrypted or not, and a detecting unit adapted to read and, if needed decrypt, the recipe code of a sample container to be processed to obtain processing information and to apply a signal to the control unit using this information. The control unit is adapted to identify the processing recipe related to the sample from the recipe code.

A specific processing recipe can be created for each designated sample processing type intended for the system, and the recipe contains parameters used in the process. The processing type relates to the type of sample to be processed, e.g. a biological sample (faeces or tissues). The processing recipe can be partly or totally stored in the control unit and partly or totally stored in the recipe code on the sample container.

A further embodiment of the system comprises an analysis unit adapted to perform analysis of the sample in accordance to control signals received from said control unit. The analysis to be performed is naturally related to the nature of the sample. Examples of different analysis are discussed above in relation to the method.

Although the present invention is particularly useful when treating biological samples (faeces or tissues), the present invention is also applicable in other fields, e.g. in the pharmaceutical industry, the food industry, the tobacco industry, etc.

In the standard setup of the invention system a weighing unit is used to automatically determine the amount of sample that is going to be processed. Often, the weight of the sample is needed to make the correct dilution in order to reach a desired final concentration range, e.g. grams of processed input sample/final milliliter of diluent. This enables that the subsequent analytical result can then be related to the amount of the analyte that is present per gram of the original sample. For instance, the analytical result relates to the level of calprotectin per gram of the faecal sample, or to the level of a protein per gram of tissue.

In one embodiment of the invention the weighing unit is not included in the automatic process. This embodiment is preferably used when the goal of the subsequent analysis step is to detect only the presence of a biomolecule, not to relate the analytical result to the amount of the biomolecule present per gram of input sample. In case of microbiology this is a common situation.

In microbiology a sample is normally taken, preferably using a swab or a brush, and the goal is to detect the presence of bacterial, viral, parasitic or fungal biomolecules such as DNA, RNA or an antigen. For instance, the sample can be from a wound or the surface of a body or a stool sample, and the goal is to analyze if MRSA bacteria are present or not.

Using the method or system according to the invention, the sample, with or without the swab or brush, is automatically homogenized in a buffer using ultrasound sonication. The sonication lyses the microorganisms of the sample and releases the biomolecules for later analysis. After the sonication, the solution is automatically centrifuged to produce a clear supernatant for subsequent analysis of the biomolecule in question. If the solution is not centrifuged it can contain large residues of the sample that hinders the subsequent analysis step to work properly. The automatic combination of ultrasound sonication and centrifugation is new.

The present invention is not limited to the above-described preferred embodiments. Various alternatives, modifications and equivalents may be used. Therefore, the above embodiments should not be taken as limiting the scope of the invention, which is defined by the appending claims.

1. Method of processing a human or animal faeces sample collected in a sample container, to be performed prior to the analysis of the sample, wherein the method comprises of automatically performing the following processing steps:
A1: providing the sample container with a recipe code and a patient ID-code related to the sample and being arranged in connection to the sample container;
A2: reading the recipe code and the patient ID-code of the sample container to obtain the relevant information for processing the sample and registration of the patient, wherein the whole or parts of a processing recipe is included in the recipe code, or as an alternative, the recipe code includes a pointer used to identify the relevant processing recipe stored in a control unit;
A3: weighing the container to determine the weight of the sample,
A4: adding a buffer where the buffer volume is predefined or related to the weight of the sample, and
A5: homogenizing and mixing of the sample in the buffer.

2. Method according to claim 1, wherein the method comprises of automatically performing the following step after A1-A5:
A6: transferring the sample container to a centrifuge and centrifuging the container in order to produce a clear supernatant,

3. Method according to claim 2, wherein the method comprises of automatically performing the following steps after A1-A6:
A7: transferring a part of the supernatant to a secondary container, the transferred volume is predefined, or related to the weight of the sample and/or related to the amount of buffer previously added, and
A8: if needed, adding buffer to the secondary container in order to dilute the sample solution to the desired final concentration.

4. Method according to claim 1, wherein in A5 the homogenizing and mixing of the sample in the buffer is performed by using sonication by ultrasound.

5. Method according to claim 1, wherein in A5 the homogenizing and mixing of the sample in the buffer is performed by using a vibrating mixing device (vortex mixer) and/or a shaking mixer device.

6. Method according to claim 1, wherein said identified processing recipe includes at least one or several of the following parameters:
a statement of the sample type; the amount of buffer to add in A4; the time for the homogenization and mixing in A5 of the sample in the buffer; the centrifugation time in A6; the amount of supernatant to withdraw in A7, the amount of buffer to add in A8; the weight of the sample container prior to sampling; the type of analysis to be performed subsequent to the automatic sample processing.

7. Method according to claim 3, when dependent upon claim 3, wherein the method comprises the following step to be performed after A8:A9: transferring a part of the solution in the secondary container to a third container for further dilution by a buffer.

8. Method according to claim 3, when dependent upon claim 3, wherein the method comprises the following step to be performed after A8 or A9, whichever is applicable:
A10: analysing the processed sample.

9. Method according to claim 1, wherein the information in the recipe code at the sample container is encrypted, and is decrypted in A2.

10. A sample processing system adapted to process a human or animal faeces sample prior to the analysis of the sample, comprising a sample container for collecting a sample, wherein the system comprises a control unit comprising a stored processing recipe that includes parameters to be used to automatically perform the processing of the sample, the control unit is adapted to generate robot control signals, in accordance with said processing recipe, to be applied to a robot arranged to move the sample container between different units of the system and to generate control signals to a specific unit when the sample container is to be treated by that unit, and that the robot is arranged to automatically move the sample container to:
a weighing unit to determine weight of the sample;
a buffer supply unit to add buffer to the sample where the buffer amount can be predefined or depend on the weight of the sample, and to
a homogenization and mixing unit to homogenize and mix the sample in the buffer.

11. Sample processing system according to claim 10, wherein the system further comprises a centrifuge to centrifuge the container in order to produce a clear supernatant.

12. Sample processing system according to claim 11, wherein the system further comprises a transfer unit in order to transfer a part of the supernatant to a secondary container, the transferred volume is predefined, or related to the weight of the sample and/or related to the amount of buffer previously added; and if needed, the buffer supply unit adds buffer to the secondary container in order to dilute the sample solution to the desired final concentration range.

13. Sample processing system according to claim 12, wherein the system further comprises a separate recipe code generation unit adapted to provide said sample container with a recipe code related to the sample, and a detecting unit adapted to read the recipe code of a sample container to be processed to obtain processing recipe information and to apply a signal to said control unit including said recipe information.

14. System according to claim 10, wherein the homogenizing and mixing of the sample in the buffer is performed by using sonication by ultrasound.

15. System according to claim 10, wherein the homogenizing and mixing of the sample in the buffer is performed by using a vibrating mixing device (vortex mixer) and/or a shaking mixer device.

16. System according to claim 13, wherein the recipe code is optically or electronically read.

17. System according to claim 16, wherein the recipe code is a bar code, or an RFID tag.

18. System according to claim 13, wherein said identified processing recipe includes at least one or several of the following parameters:
a statement of the sample type; the amount of buffer to add; the time for the homogenization and mixing of the sample in the buffer; the centrifugation time; the amount of supernatant to withdraw; the weight of the sample container prior to sampling; the type of analysis to be performed subsequent to the automatic sample processing.

19. System according to claims 10, wherein the system comprises an analysis unit adapted to perform analysis of the sample, in accordance to control signals received from said control unit.

20. System according to claim 13, when dependent upon claim 13, wherein the information in said recipe code is encrypted, and that said detecting unit is adapted to decrypt the read recipe code.
